(19) United States
(12) Patent Application Publication
(10) Pub. No.: US 2024/0148440 A1
(43) Pub. Date: May 9, 2024

(54) MEDICAL SYSTEM, AND METHOD FOR CHECKING THE COMPATIBILITY OF IMPLANTS AND INSTRUMENTS OF A MEDICAL SYSTEM

(71) Applicant: Aesculap AG, Tuttlingen (DE)

(72) Inventors: Thomas Pleil, Bad Duerrheim (DE); Thomas Scholten, Tuttlingen (DE); Michael Willmann, Villingen-Schwenningen (DE); Alexander Happle, Huefingen (DE); Jens Beger, Tuttlingen (DE); Pirmin Lutz, Muehlingen (DE)

(21) Appl. No.: 18/412,928

(22) Filed: Jan. 15, 2024

Related U.S. Application Data
(63) Continuation of application No. PCT/EP2022/069881, filed on Jul. 15, 2022.

(30) Foreign Application Priority Data
Jul. 16, 2021 (DE) 10 2021 118 400.2

(51) Int. Cl. A61B 34/10; A61B 90/00; A61B 90/94

(52) U.S. Cl. CPC A61B 34/10; A61B 90/94; A61B 2034/108; A61B 2090/0807

(57) ABSTRACT

A medical system includes at least two mutually different implants and at least two mutually different instruments. Each of the instruments is configured and intended to cooperate and be compatible with an implant. The medical system also includes a microscope that defines a microscope field of view and a compatibility checking apparatus. The compatibility checking apparatus can be used in a method to automatically check for compatibility between an instrument and an implant when the instrument and the implant are positioned together in the microscope field of view.

### CROSS-REFERENCE TO RELATED APPLICATIONS

This application is filed under 35 U.S.C. § 365(c) as a continuation of International Application No. PCT/EP2022/069881, filed on Jul. 15, 2022, and claims priority to German Application No. 10 2021 118 400.2, filed on Jul. 16, 2021. The contents of International Application No. PCT/EP2022/069881 and German Application No. 10 2021 118 400.2 are incorporated by reference herein in their entireties.

### FIELD

The present disclosure relates to a medical system comprising at least two mutually different implants and at least two mutually different instruments, wherein each of the at least two mutually different instruments is configured and intended to cooperate with an implant of the at least two mutually different implants that is compatible and associated therewith.

The present disclosure further relates to a method for automatically checking a compatibility of implants and instruments of a medical system comprising at least two mutually different implants and at least two mutually different instruments, wherein each of the at least two mutually different instruments is configured and intended to cooperate with an implant of the at least two mutually different implants that is compatible and associated therewith.

### BACKGROUND

Medical systems of the aforementioned type are used, in particular, in neurosurgery. They comprise a plurality of different medical clips in the form of aneurysm clips. In particular, the clips are differently formed for different application cases, have different closing forces and are made from different materials, for example, titanium or an alloy marked under the registered trademark PHYNOX®. Since the clips are delicate and for a reliable implantation cannot be applied in an undefined manner with any desired instrument, for example a pair of forceps or pliers, in particular different instruments are utilised dependent upon and in cooperation with the clips, some of which, as described, are very different. It is therein important that the instrument and the clip are mutually compatible, in order to avoid damage to the respective clip by the instrument when cooperating.

### SUMMARY

It is therefore an object of the present disclosure to improve a medical system such that it can be reliably utilized.

This object is achieved with a medical system of the type mentioned in the introduction in accordance with the present disclosure in that the medical system comprises a microscope which defines a microscope field of view, and a compatibility checking apparatus for automatic checking of a compatibility of one of the at least two mutually different instruments and one of the at least two mutually different implants, which are positioned together in the microscope field of view.

The proposed development of a known medical system makes it possible, in particular, for a user to check reliably in a simple way whether the instrument he uses for the implantation and the implant intended for the implantation are compatible with one another. The system performs the checking automatically. For this purpose, the instrument and the implant must only be positioned in the microscope field of view together. The compatibility checking apparatus can then recognize the implant and the instrument, for example, from their respective shapes and/or sizes or an identification characterising the implant and/or the instrument, for example, a code. It should be noted herein that in medical systems in which implants are implanted with the aid of a microscope, a surgeon often receives an instrument handed to him already in engagement with the implant that is to be implanted. Since he observes only the microscope image during the intervention and his gaze remains on the operation site, he has no possibility himself of checking, with a direct view of the unit handed to him, whether the instrument and the implant held therein are compatible or not. The proposed system enables him, in particular, when he brings the implant and the instrument into the microscope field of view, to check it by means of the system, specifically in an automated manner. Thus, before a final implantation of the relevant implant, it can be ensured, in particular, whether or not the combination of instrument and implant handed to the surgeon by an assisting person are indeed intended for a cooperation.

It is favourable if the microscope is configured in the form of an optical microscope or a digital microscope. It is ultimately unimportant for the performance of the compatibility check what type the microscope is. It is carried out automatically with the compatibility checking apparatus of the system. Thus, for example, medical systems that are already in use can easily be retrofitted.

It is advantageous if the microscope comprises an optical microscope image sensor. In particular, this can be configured in the form of a CCD sensor or a CMOS sensor. Microscope image sensors of these types can be used particularly in digital microscopes. They can however also be used in optical microscopes to which a digital camera can be connected. In this way, for example, purely optical microscopes can be retrofitted. Such optical microscope image sensors allow a digital image of the microscope field of view to be generated, stored and further processed. This can then optionally be used to read out data, for example, inscriptions and codes on the implant or instrument that are visible in the microscope field of view.

According to a further preferred embodiment, it can be provided that the compatibility checking apparatus comprises an optical detection device for detecting an optical code, that the detection device defines a detection field of view and that the microscope field of view and the detection field of view at least partially overlap. The optical detection device enables, in particular, an image of the detection field of view to be generated. If the detection field of view and the microscope field of view overlap, then at least a portion of the microscope field of view can be captured with the detection device. If, for example, an optical microscope is used which comprises no microscope image sensor, that is, no digital image capturing device, an image of the detection field of view can be acquired with the optical detection device and, as a result of the proposed arrangement, at least a portion of the microscope field of view can also be acquired. It is thus possible to retrofit conventional medical systems, for example, to equip optical microscopes with a compatibility checking apparatus.

In order to be able to check the compatibility of the instrument and the implant reliably, it is advantageous if an overlap of the microscope field of view and the detection field of view is in a range from approximately 50% to 100%. In particular, the overlap can be in a range from approximately 80% to 100%. Preferably, the overlap is in a range from approximately 95% to 100%. The larger the overlap of the microscope field of view and the detection field of view is, the more reliably all the objects in the microscope field of view can be captured and checked for compatibility. Ideally, the overlap is large enough so that all the parts situated in the microscope field of view are also reliably recognisable in the detection field of view.

In order to be able to evaluate an image of the detection field of view rapidly and simply, it is advantageous if the optical detection device comprises at least one optical image sensor. In particular, the optical image sensor can be a CCD sensor or a CMOS sensor. The optical image sensor can be included, for example, by a camera which is connected to an optical microscope. As mentioned, optical microscopes can thus be easily retrofitted. In addition, digital recordings can be easily evaluated, for example, objects or codes therein can be easily identified.

A particularly compact medical system can be provided if the microscope image sensor defines or forms the optical image sensor. In this way, it can be achieved, in particular, that the detection field of view and the microscope field of view overlap completely, that is by 100%. In other words, in this case, the compatibility checking apparatus uses the microscope image sensor to generate the detection field of view, which coincides with the microscope field of view.

It is favourable if the at least two mutually different implants and the at least two mutually different instruments each comprise at least one optical code, that the optical code of the at least two mutually different implants differs and that the optical code of the at least two mutually different instruments differs. Encoding implants and instruments in the manner described enables a user to distinguish them in a targeted manner Thereby, in particular, by means of the optical code, the size or, for example, the jaw geometry of an instrument can be encoded. In the case of implants, for example, medical clips, their size and, for example, also their closing forces and the materials from which they are formed, can thus be encoded. By means of optical codes, a simple and possibly redundant compatibility check can also be carried out by sight, for example, if the optical codes are simple colour codes. Thus, for example, a person assisting a surgeon can recognise immediately whether the instrument and the implant are compatible, for example, by comparison of a colour code of the instrument and the implant.

It is favourable if the at least one optical code of implants and instruments that are compatible with one another and are intended for cooperation together, is identical. Thus, independently of the automatic compatibility check by the system, a user can also carry out a simple visual check, purely optically, by observing the instrument and the implant, and can thus recognise directly whether the combination of instrument and implant is at all compatible, for example, is permitted by a manufacturer. However, it is not absolutely required that mutually compatible instruments and implants have an identical code. A compatibility can also occur, in particular, with different codes and a compatibility check can take place by comparison of these codes, the compatibility of which is stored in corresponding tables.

In a simple manner, instruments and implants can be automatically recognised if the at least one optical code is configured in the form of a bar code, a data matrix code, a QR code, a shape code or a colour code. Thus, for example, from digital acquisitions of the detection field of view, bar codes, a data matrix code, a QR code, a shape code or a colour code can be easily recognised. Shape codes can be defined, in particular, by the shape of the implants and/or instruments themselves, or at least a portion thereof. In this case, no additional code is absolutely necessary. The code is defined by the implant and the instrument themselves. Each implant and/or each instrument can also comprise two or more optical codes. A combination, in particular, of the aforementioned code forms can herein be involved. The at least one optical code, in particular a data matrix code and/or the other alternatives of optical codes mentioned above can also be used as markers for navigation applications and/or for augmented reality applications during implantation of the implant, in particular, during the application of a medical clip. In particular, non-visible structures can be represented, for example, the implant and the associated instrument. In particular, the medical clip, an associated clip application instrument and a vessel structure to which the clip is to be applied can be represented. It is further possible, in particular, to assess cooperating elements, in particular, mutually abutting surface regions of, for example, a medical clip and the clip application instrument with regard to a seating of the clip in a clip receptacle, also designated the jaw portion of the clip application instrument, by means of a shape recognition.

Preferably, the compatibility checking apparatus comprises an image processing device for automatically determining instruments and/or implants in the microscope field of view. The image processing device can, in particular, also be configured for automatically determining instruments and/or implants in the detection field of view. The image processing device enables, in particular, optical codes in a recorded image of the microscope field of view and/or the detection field of view to be evaluated by simple means. This offers possibilities, in particular, for process optimisation, for example with regard to a documentation of a surgical intervention and a follow-up of implants and instruments used, also designated as tracking.

It is favourable if the image processing device is configured for reading out the at least one code of instruments and/or implants from an image of the detection field of view and/or the microscope field of view. An image processing device of this type enables, in particular, instruments and implants to be recognised from their code and possibly also to indicate to a user which instrument and/or implant is involved. Codes can also be easily extracted from an image by comparison with stored codes.

Preferably, the compatibility checking apparatus comprises a display device for displaying a result of the compatibility check. This can entail, for example, an image screen. The display device can also be configured, in particular, in the form of an acoustic display device in order to acoustically communicate to the user whether an implant and an instrument are compatible or not. The display device can be configured, in particular, in the form of a display of the microscope or in the form of an external display independent of the microscope. For example, in a hybrid operating theatre, the display device can be included by a pair of data goggles or can comprise a speech output.

It is advantageous if the microscope comprises a microscope display device for displaying an image of the microscope field of view and if the microscope display device comprises the display device. The medical system can thus be configured particularly compact. For example, the display device can be configured in the form of a window or subregion of the microscope display device. Therefore, if required, results of compatibility checks can be superimposed onto a microscope image displayed on the microscope display device. Thus, a surgeon who sees on the microscope display device, for example, an image of the microscope field of view, that is, for example the operation site, can receive it, directly superimposed, whether the instrument shown and the implant thereby held are compatible.

According to a further preferred embodiment, it can be provided that the at least two implants are configured in the form of medical clips and that the at least two instruments are configured in the form of clip application instruments. In particular, the medical clips can be configured in the form of aneurysm clips. The clip application instruments, also known as applicators, cooperate with the medical clips in order to apply them, for example, for treating bulgings on hollow organs in the body of a person or an animal.

It is favourable if the at least two instruments comprise an instrument end that is bringable into engagement with one of the at least two implants and if the at least one optical code is arranged or configured on or in the region of the instrument end. If the instrument end of the instrument which is positioned in the microscope field of view is in engagement with an implant, then the codes both on the implant and also on the instrument can be positioned in close spatial proximity to one another and thereby easily captured.

The object set out in the introduction is further achieved with a method of the type stated in the introduction in accordance with the present disclosure in that the compatibility of an implant and an instrument of the medical system is checked automatically when the implant and the instrument are positioned together in the microscope field of view of a microscope.

As explained in detail above, an additional assurance can thereby be achieved when instruments and implants are used, since whether instruments and implants are mutually compatible cannot necessarily be recognised by a user at first sight in all cases. A method of this type therefore helps to prevent the implantation of particular implants with instruments not intended for them.

It is favourable if the implants and instruments of the system have an optical code and if the compatibility is checked by a comparison of the optical code of the implant and the optical code of the instrument which are positioned together in the microscope field of view. A procedure of this type does not necessarily require the provision of code tables. In particular, new instruments and implants which have not previously been stored in a code table can thus also be checked for compatibility. A compatibility can exist, in particular, if the optical codes of the instrument and the implant are identical.

For a reliable use of the method, it is advantageous if the result of the comparison is output. In particular, this can take place optically and/or acoustically. For example, an incompatibility can be notified to a surgeon by a warning sound or a suitable announcement.

Furthermore, the use of one of the above-described systems for carrying out one of the aforementioned methods is proposed.

### BRIEF DESCRIPTION OF THE DRAWINGS

The following description of preferred embodiments of the present disclosure, in conjunction with the drawings, provides a more detailed explanation. In the drawings:

FIG. 1 shows a schematic overview of an exemplary embodiment of a medical system;

FIG. 2 shows a schematic representation of the functioning of an exemplary embodiment of a medical system;

FIG. 3 shows a schematic representation of the functioning of a further exemplary embodiment of a medical system;

FIG. 4 shows a schematic representation of an overlap of a microscope field of view and a detection field of view;

FIG. 5 shows a schematic representation of an instrument and an implant that are mutually compatible; and

FIG. 6 shows a schematic representation of an instrument and an implant that are not mutually compatible.

### DETAILED DESCRIPTION

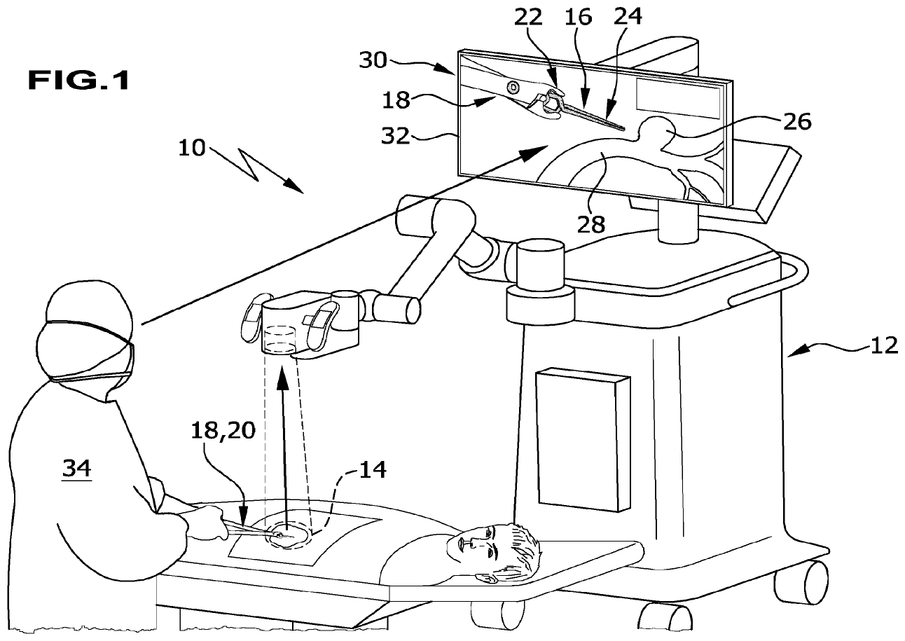

An exemplary embodiment of a medical system 10 is represented schematically in FIG. 1.

It comprises a microscope 12 which defines a microscope field of view 14. The system further comprises two or more mutually different implants 16 and two or more mutually different instruments 18. The instruments 18 and implants 16 are configured and intended, in the event of compatibility, to cooperate with one another as intended.

Represented schematically in FIG. 1 is an example of an instrument 18 in the form of a clip applicator 20 which holds at a distal instrument end 22 an implant 16, in the form of a medical clip 24, that has been received therein. With the clip applicator 20, the medical clip 24 can be opened and, for example, applied to a bulging 26, also referred to as an aneurysm, of a hollow organ, in particular in the form of a blood vessel 28.

As shown schematically in FIG. 1, the microscope 12 is configured in the form of a digital microscope. It comprises a microscope display device 30 in the form of a screen 32 on which an image of the microscope field of view 14 is represented. A surgeon 34 who handles the instrument 18 monitors the application of the implant 16 on the screen 32.

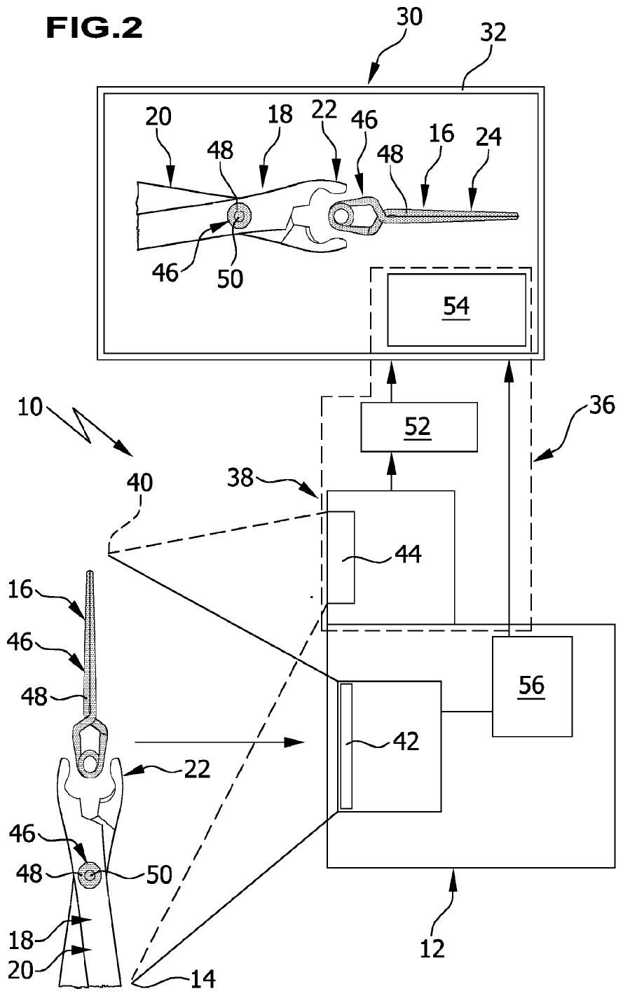

FIG. 2 shows schematically an exemplary embodiment of a medical system 10 which comprises a compatibility checking apparatus 36. It serves the purpose of automatically checking a compatibility of the instrument 18 and the implant 16 which are positioned together in the microscope field of view 14.

The compatibility checking apparatus 36 comprises an optical detection device 38. It defines a detection field of view 40. The detection device 38 provided in this exemplary embodiment independently of the microscope 12 is arranged such that the microscope field of view 14 and the detection field of view 40 overlap by at least half. Therefore, an overlap of the microscope field of view 14 and the detection field of view 40 is in the region of at least approximately 50%.

The microscope 12 represented schematically in FIG. 2 comprises an optical microscope image sensor 42. This is configured in the form of a CCD sensor or a CMOS sensor.

The optical detection device 38 comprises an optical image sensor 44. This is configured as a CCD sensor or a CMOS sensor.

The mutually different implants 16 and the mutually different instruments 18 of the medical system 10 each comprise an optical code 46. It is configured in the form of a colour code 48. In the exemplary embodiment of FIG. 2, the implant 16 is coloured. On the instrument 18, an element of the instrument 18 arranged in the region of the instrument end 22 is identified with a colour code 48. In the exemplary embodiment of FIG. 2, the element is a locking screw 50 of the instrument 18 configured as a clip applicator 20.

The detection device 38 is configured to detect the optical code 46. This is enabled in that the detection device 38 acquires an image of the detection field of view 40 with the image sensor 44. The compatibility checking apparatus 36 comprises an image processing device 52. With this, instruments 18 and implants 16 can be captured in the detection field of view 40 and in the region of the microscope field of view 14 that overlaps with the detection field of view 40.

In the exemplary embodiment of FIG. 2, the image processing device 52 is configured to read out the optical code 46 of the instrument 18 and of the implant 16 from the image of the detection field of view 40. Therefore, the image processing device 52 is configured to recognise the colour code 48 on the instrument 18 and the implant 16.

In order to display a result of the compatibility check, the compatibility checking apparatus 36 comprises a display device 54. In the exemplary embodiment of the medical system 10 shown in FIG. 2, the microscope display device 30 comprises the display device 54. This is realised in the form of a superimposed window in the image of the microscope field of view 14 displayed on the screen 32. It is shown on the display device 54 in a manner that is not shown, in text form or, for example, with a traffic-light representation, whether the colour code 48 of the instrument 18 and the implant 16 match. In the exemplary embodiment shown in FIG. 2, this is the case.

The image of the microscope field of view 14 created with the microscope image sensor 42 is processed with a computing device 56 and displayed on the microscope display device 30.

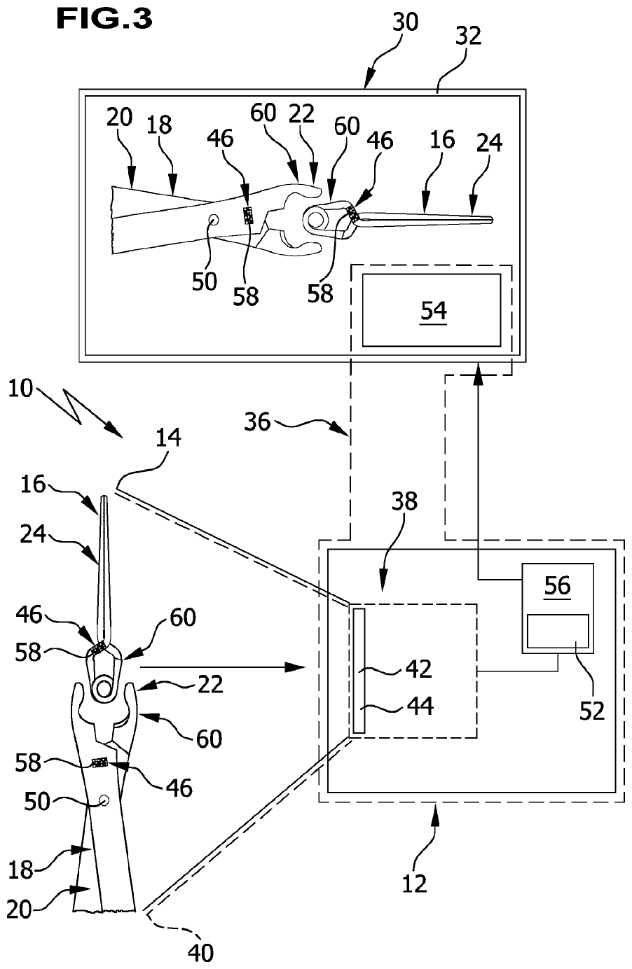

A further exemplary embodiment of a medical system 10 is represented schematically in FIG. 3. In this exemplary embodiment, the optical detection device 38 is incorporated by the microscope 12. The microscope image sensor 42 forms the optical image sensor 44.

The compatibility checking apparatus 36 comprises, in particular, the microscope 12 and the display device 54 included by the microscope display device 30. The image of the microscope field of view 14 created with the microscope image sensor 42 is processed with an image processing device 52 incorporated by the computing device 56 and displayed on the microscope display device 32. The result of the compatibility check is displayed on the screen 32, in particular as described above in relation to the exemplary embodiment of FIG. 2, in the form of a descriptive text and/or in the form of a traffic-light representation.

In the schematic exemplary embodiment shown in FIG. 3, an optical code 46 is provided on the instrument 18 and the implant 16, specifically in the form of a data matrix code 58.

The image processing device 52 is configured to extract the optical codes 46 of the instrument 18 and the implant 16 from the image of the microscope field of view 14 acquired by the microscope image sensor 42 and to compare them with one another.

Both the exemplary embodiment of FIG. 2 and the exemplary embodiment of FIG. 3 of the medical system are optionally configured to extract a shape code from the image of the microscope field of view 14 and/or of the detection field of view 40. The shape code 60 is defined by the implant 16 per se and by the instrument 18 per se. In particular, both exemplary embodiments can also recognise different codes 46 on an implant 16 and on an instrument 18. This also allows a more reliable checking of the implant 16 and the instrument 18 since, effectively during the checking of two optical codes 46 both on the instrument 18 and on the implant 16, a redundant compatibility information item can be obtained.

In the exemplary embodiment of FIG. 3, the optical code 46 is arranged distally of the locking screw 50 on the instrument 18 and therefore in the region of the instrument end 22.

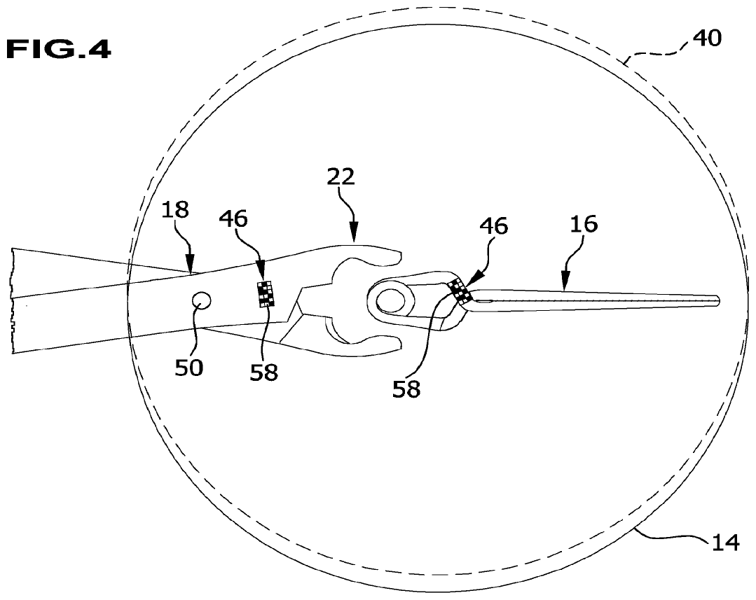

FIG. 4 shows schematically the overlapping of the microscope field of view 14 and the detection field of view 40. In order to be able to carry out the compatibility check, the implant 16 and the instrument 18 must be positioned in the microscope field of view 14 and simultaneously in the detection field of view 40 such that both the optical codes 46, that is, both on the implant 16 and also on the instrument 18 can be captured simultaneously. This enables both the captured optical codes 46 to be compared with the compatibility checking apparatus 36 and a compatibility of the implant 16 and the instrument 18 to be confirmed automatically if the two optical codes 46 are identical, or to output a warning if the two optical codes 46 differ.

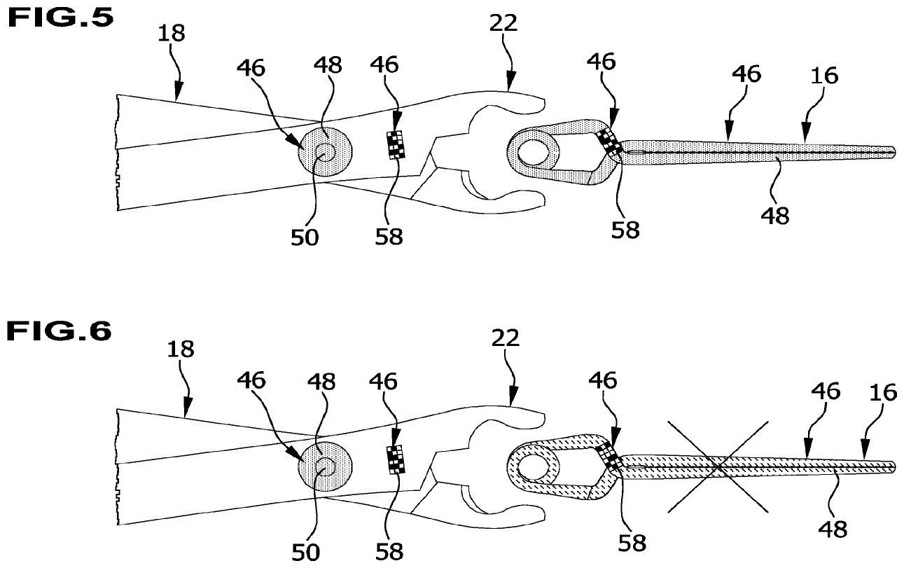

In FIGS. 5 and 6, these two alternatives, that is, compatibility and incompatibility of the implant 16 and the instrument 18 are represented schematically.

In FIG. 5, both on the instrument 18 and also on the implant 16 respectively, two optical codes 46 are provided, specifically one as a colour code 48 and one as a data matrix code 58. In addition, the shape of the instrument 18 and of the implant 16 can optionally be captured and checked for compatibility.

In the example of FIG. 5, the two optical codes 46 match. In the example of FIG. 6, both the colour codes 48 and the data matrix codes 58 differ. Therefore, in the situation according to FIG. 5, the compatibility of the instrument 18 and the implant 16 can be confirmed but in the example of FIG. 6, it is denied. For example, a warning can then be output.

In alternative exemplary embodiments which are not shown, further optical codes in the form of bar codes or QR codes can alternatively or additionally be provided.

In the optical code 46, in particular, a size of the implant, for example, three different size families, specifically “standard”, “mini” and “long” can be differentiated. To these three size families, there correspond three associated instruments 18 which are compatible. For example, the three sizes can be encoded by means of a suitable colour code, for example, blue for “standard”, red for “mini” and green for “long”.

A colour code can be used, in particular, to differentiate implants remaining lastingly in the body, for example, permanent clips of an aneurysm clip system or temporary implants, for example, temporary clips of such an aneurysm clip system from one another. In particular, in addition to the coloured code for the size, a coloured code for the information “permanent” or “temporary” can be supplemented.

With the data matrix code, different information items regarding the instrument 18 and the implant 16 can be encoded and stored directly on the instrument 18 and/or on the implant 16, for example, the manufacturing date, the closing force of a clip or the material from which the clip has been made. The information encoded in the data matrix code can also be used, in particular, for tracking and documenting a surgical intervention. Therefore, with the exemplary embodiments of medical systems 10 as described, not only can compatibilities of instruments 18 and implants 16 be checked, but the information established can also be used for process optimisation. It is, for example, possible to trigger a re-order process automatically when a particular implant 16 has been implanted. In addition, implanted implants can also be allocated directly to a patient identification card. Furthermore, it can be established how often which type of implant 16 has been implanted permanently and which temporary implants 16 have been used.

Implants 16 and instruments 18 can be tracked with the exemplary embodiments of medical systems 10 described, in particular also intraoperatively, and in the preparation process. This represents a service to the user, offers a data collection system for the manufacturer and enables a capture of register data and the use in clinical studies.

In the exemplary embodiment of FIG. 2, the detection device 38 is configured separately from the microscope 12. It can be realised in the form of any desired digital camera. For example, in an operating room, a navigation camera or a pair of data goggles with an integrated camera can be used for this purpose.

Alternatively, for the integration of the display device 54 into the microscope display device 30, an additional screen can be provided. The result of the compatibility check can also be displayed in a pair of data goggles which a surgeon wears, or can take place acoustically by means of a speech output.

The exemplary embodiments of medical systems 10 as described easily enable an automatic check of whether an instrument 18 and an implant 16 which should be used together are compatible with one another or not. This information is of great consequence since thereby, in particular, damage to implants 16 through the use of an incompatible instrument 18 can be avoided.

What is claimed:

1. A medical system comprising:
at least two mutually different implants;
at least two mutually different instruments;
a microscope; and
a compatibility checking apparatus,
each of the at least two mutually different instruments being configured to cooperate with one of the at least two mutually different implants that is compatible,
the microscope defining a microscope field of view,
the compatibility checking apparatus being configured for automatic checking of a compatibility of one of the at least two mutually different instruments and one of the at least two mutually different implants that are positioned together in the microscope field of view.

2. The medical system according to claim 1, wherein the microscope is an optical microscope or a digital microscope.

3. The medical system according to claim 1, wherein the microscope comprises an optical microscope image sensor.

4. The medical system according to claim 3, wherein:
the compatibility checking apparatus comprises an optical detection device for detecting an optical code,
the detection device defines a detection field of view, and
the microscope field of view and the detection field of view at least partially overlap.

5. The medical system according to claim 4, wherein an overlap of the microscope field of view and the detection field of view is in a range of approximately 50% to approximately 100%.

6. The medical system according to claim 4, wherein the optical detection device comprises at least one optical image sensor.

7. The medical system according to claim 6, wherein the optical microscope image sensor defines or forms the optical image sensor.

8. The medical system according to claim 1, wherein:
the at least two mutually different implants and the at least two mutually different instruments each comprise at least one optical code,
each optical code of the at least two mutually different implants being different, and
each optical code of the at least two mutually different instruments being different.

9. The medical system according to claim 8, wherein optical codes of implants and instruments that are compatible with one another are identical.

10. The medical system according to claim 8, wherein the at least two mutually different instruments comprise an instrument end that is engageable with one of the at least two mutually different implants, and wherein the at least one optical code is arranged or configured on or in the region of the instrument end.

11. The medical system according to claim 8, wherein each of the optical codes is a bar code, a data matrix code, a QR code, a shape code or a color code.

12. The medical system according to claim 10, wherein the image processing device is configured for reading out the at least one code of instruments and/or implants from an image of the detection field of view and/or the microscope field of view.

13. The medical system according to claim 1, wherein the compatibility checking apparatus comprises an image processing device for automatically determining instruments and/or implants in the microscope field of view.

14. The medical system according to claim 1, wherein the compatibility checking apparatus comprises a display device for displaying a result of the compatibility check.

15. The medical system according to claim 14, wherein:
the microscope comprises a microscope display device for displaying an image of the microscope field of view, and
the microscope display device comprises the display device.

16. The medical system according to claim 1, wherein the at least two mutually different implants are medical clips, and wherein the at least two mutually different instruments are clip application instruments.

17. A method for checking compatibility of implants and instruments of a medical system that has at least two mutually different implants and at least two mutually different instruments, with each of the at least two mutually different instruments configured to cooperate and be compatible with one of the at least two mutually different implants, the method comprising the steps of:
positioning one of the at least two mutually different instruments adjacent to said one of the at least two mutually different implants; and
automatically checking compatibility of said one of the at least two mutually different instruments with said one of the at least two mutually different implants when said one of the at least two mutually different instruments and said one of the at least two mutually different implants are positioned together in a microscope field of view of a microscope.

18. The method according to claim 17, wherein the step of automatically checking compatibility comprises a comparison step in which a first optical code on said one the at least two mutually different implants is compared with a second optical code on said one of the at least two mutually different instruments.

19. The method according to claim 18, further comprising the step of outputting a result of the comparison step.

20. The method according to claim 19, wherein the result of the comparison step is output optically and/or acoustically.
